SEQUENCE LISTING (2 sequences)

SEQ ID NO: 1 - DNA, 27 bp, Mycobacterium avium subspecies paratuberculosis
gaagggtgttcggggccgtcgcttagg

SEQ ID NO: 2 - DNA, 27 bp, Mycobacterium avium subspecies paratuberculosis
ggcgttgaggtcgatcgcccacgtgac

(19) United States
(12) Patent Application Publication
(10) Pub. No.: US 2009/0054380 A1
(43) Pub. Date: Feb. 26, 2009

(54) Methods for diagnosing and treating a mycobacterium avium subspecies paratuberculosis infection

(76) Inventor: Robert J. Greenstein, Tenafly, NJ (US)

Correspondence Address: Jane Massey Licata;Licata & Tyrrell P.C., 66 East Main Street, Marlton, NJ, 08053, US

(21) Appl. No.: 12/119,657

(22) Filed: May 13, 2008

Related U.S. Application Data
(63) Continuation-in-part of application No. 11/831,037, filed on Jul. 31, 2007.
Application No. 11/831,037 is a continuation-in-part of application No. PCT/US2006/062316, filed on Dec. 19, 2006.
(60) Provisional application No. 60/751,957, filed on Dec. 20, 2005.
(60) Provisional application No. 60/779,541, filed on Mar. 6, 2006.
(60) Provisional application No. 60/745,439, filed on Apr. 24, 2006.
(60) Provisional application No. 60/806,007, filed on Jun. 28, 2006.
(60) Provisional application No. 60/821,734, filed on Aug. 8, 2006.
(60) Provisional application No. 60/822,442, filed on Aug. 15, 2006.

(51) Int. Cl. A61K 31/60; A61K 31/454; A61P 31/06; C12Q 1/04; C12Q 1/18

(52) U.S. Cl. 514/161; 514/323; 514/159; 435/34; 435/32

(57) ABSTRACT

The present invention relates to Mycobacterium avium subspecies paratuberculosis (MAP) as the etiological agent of IBD, including ulcerative colitis and Crohn's disease, as well as Multiple Sclerosis and Alzheimer's Disease. As such, the present invention provides a method for diagnosing Multiple Sclerosis and Alzheimer's Disease by detecting the presence of MAP nucleic acid molecules as well as methods for inhibiting the proliferation of MAP and treating a MAP infection using anti-MAP agents. Pharmaceutical compositions containing anti-MAP agents and methods for determining the antibiotic susceptibility of MAP isolated from a subject are also provided.

### INTRODUCTION

This application is a continuation-in-part application of U.S. patent application Ser. No. 11/831,037 filed Jul. 31, 2007, which is a continuation-in-part application of PCT/US2006/062316, filed Dec. 19, 2006, which claims benefit of U.S. Provisional Patent Application Ser. Nos. 60/751,957 filed Dec. 20, 2005, 60/779,541 filed Mar. 6, 2006, 60/745,439 filed Apr. 24, 2006, 60/806,007 filed Jun. 28, 2006, 60/821,734 filed Aug. 8, 2006 and 60/822,442 filed Aug. 15, 2006, the contents of which are incorporated herein by reference in their entireties.

### BACKGROUND OF THE INVENTION

Johne's disease is a chronic diarrheal enteric disease in ruminants that is caused by Mycobacterium avium subspecies paratuberculosis (MAP)(Johne & Frothingham (1895) Dtsch. Zeitschr. Tiermed. Vergl. Pathol. 21:438-454). Live MAP is shed into the milk of cows with Johne's disease (Sweeney (1996) Vet. Clin. North Am. Food Anim. Pract. 12(2):305-12). MAP has been cultured from commercially available pasteurized milk in Europe and the United States (Grant (1998) Appl. Environ. Microbiol. 64(7):2760-1; Ellingson, et al. (2005) J. Food Prot. 68(5):966-72). When the entity at present know as Crohn's disease was first described (Crohn, et al. (1932) J. Amer. Med. Assoc. 99:1323-1328), similarities to Johne's disease were identified (Dalziel (1913) Br. Med. J. ii:1068-1070). However, in humans MAP exists in the cell wall-deficient form (Chiodini (1987) J. Clin. Microbiol. 25:796-801). Therefore, in the early analysis of Crohn's disease, MAP could not be detected in humans by the mycobacterial identification techniques of the time, because such techniques stained the mycobacterial cell wall (Ziehl (1882) Dtsch. Med. Wschr. 8:451; Neelsen (1883) Zbl. Med. Wiss. 21:497-501). However, since 1913 the presence of MAP has been identified in humans by other means (see, e.g., Greenstein (2003) Lancet Infect. Dis. 3(8):507-14) and an infectious etiology has been posited for some (Hermon-Taylor (1998) Ital. J. Gastroenterol. Hepatol. 30(6):607-10; Borody, et al. (2002) Dig. Liver Dis. 34(1):29-38), or all (Greenstein (2005) Genetics, Barrier Function, Immunologic & Microbial Pathways. Munster, Germany:25) of inflammatory bowel disease (IBD).

Since the first detection of MAP RNA (Mishina, et al. (1996) Proc. Natl. Acad. Sci. USA 93(18):9816-9820), MAP has been suggested as being the primary and unique, etiological agent of all IBD (Naser, et al. (2004) Lancet 364(9439):1039-1044; Autschbach, et al. (2005) Gut 54(7):944-9; Greenstein (2005) supra; Greenstein (2005) Genetics, Barrier Function, Immunologic & Microbial Pathways. Munster, Germany:24; Greenstein (2005) Crohn's and Colitis Foundation (CCFA) National Research and Clinical Conference. Fourth Annual Advances in Inflammatory Bowel Disease. Miami, Fla.:211) including Perforating and Non-perforating Crohn's disease (Greenstein, et al. (1988) GUT 29:588-592; Gilberts, et al. (1994) Proc. Natl. Acad. Sci. USA 91(126):12721-12724) and ulcerative colitis. It is posited that the particular clinical presentation of IBD that manifests is dependent upon the infected individual's immune response to MAP (Gilberts, et al. (1994) supra). This is analogous to another mycobacterial disease, leprosy. There are two clinical forms of leprosy, tuberculoid and lepromatous (Hansen (1874) Norsk Magazin Laegevidenskaben 4:1-88), both of which are caused by the same organism, M. leprae. The form of leprosy that manifests in a given individual is determined by the immune response of the infected patient (Yamamura, et al. (1991) Science 254:277-279), not by the phenotype or genotype of the leprosy bacillus.

It has been suggested that Koch's postulates (Koch (1882) Berl. Klin. Wschr. 19:221-230), originally promulgated for use in demonstrating tuberculosis infection, may have been met for MAP in Crohn's disease (Greenstein (2003) supra) and more recently for MAP in ulcerative colitis (Greenstein (2005) supra; Naser, et al. (2004) supra).

The link between MAP infection and other diseases has not been as extensively analyzed. An association between ulcerative colitis and Multiple Sclerosis has been suggested (Rang, et al. (1982) The Lancet pg. 555) and the positive association between IBD incidence rates and Multiple Sclerosis has led to the suggestion that these two chronic, immunologically-mediated diseases may have a common environmental etiology (Green, et al. (2006) Am. J. Epidemiol. 164(7):615-23). However, the common causal agent of ulcerative colitis and Multiple Sclerosis was not identified. Moreover, while the symptoms of Multiple Sclerosis have been ameliorated with variety of therapeutic agents including azathioprine, methotrexate, cyclophosphamide and mitoxantrone (Kaffaroni, et al. (2006) Neurol. Sci. 27 Suppl. 1:S13-7), which have been suggested to mediate the secondary inflammatory response, there has been no indication that these agents affect the primary etiological agent.

Sulfasalazine, developed in 1942, has been used to treat rheumatoid arthritis and ulcerative colitis (Svartz (1942) Acta Medica Scandinavica 110:577-598). Sulfasalazine has become, because of empirically observed clinical efficacy, the most common medicine used to treat IBD (Berardi (1997) In: Herfindal E T, Gourley D R, eds. Textbook of Therapeutics. Drugs and Disease Management. Baltimore: Williams and Wilkins, pp. 483-502), with greatest efficacy in ulcerative colitis (Travis (2002) Gut 51(4):548-9; Green, et al. (1998) Aliment. Pharmacol. Ther. 12(12):1207-16; Stenson & Korznik (2003) In: Yamada T, ed. Textbook of Gastroenterology. 4th ed. Lippincott Williams & Wilkins, Philadelphia, Pa., pp. 1727-1828). Sulfasalazine is a conjugate composed of 5-aminosalicylic acid (5-ASA) and sulfapyridine. It is generally accepted that the active moiety of sulfasalazine is 5-ASA (Azad, et al. (1977) Lancet 2:829-31).

5-Aminosalicylic acid (5-ASA, a component of sulfasalazine, olsalazine and balsalazide), and 5-ASA derivatives such as 4-aminosalicylic acid (4-ASA), have been used in the treatment of all forms of IBD, with greatest efficacy in ulcerative colitis (Stenson & Korznik (2003) Textbook of Gastroenterology. Fourth Ed., Lippincott Williams & Wilkins, Philadelphia, Pa. pg. 1727-1828; Schreiber, et al. (1994) Gut 34:1081-1085). 4-aminosalicylic acid, and its derivatives, exhibit tuberculostatic activity (Ertan, et al. (1985) Mikrobiyol. Bul. 19:121-6; Brown & Ratledge (1975) Biochim. Biophys. Acta 385:207-20) and have been suggested for use as adjunct therapeutics to anti-atypical mycobacterial agents such as clarithromycin, rifabutin, and rifampicin (U.S. Pat. No. 6,551,632). Similarly, aspirin (acetylsalicylic acid) and salicylic acid are known to reduce bacterial titers in experimental endocarditis (Nicolau, et al. (1993) Infect. Immun. 61:1593-5; Kupferwasser, et al. (2003) J. Clin. Invest. 112(2):222-33), and 2-(p-aminobenzenesulphonamido) pyridine has antibacterial efficacy in vitro (Whitby (1938) Lancet 1:1210-1212) and has been used as an antibiotic clinically (Evans & Gaisford (1938) Lancet 2:14-19). However, anti-MAP activity of 5-ASA and other salicylic acid derivatives as a primary therapeutic in the treatment of IBD has not been posited or demonstrated. Rather, the effect of 5-ASA in the treatment of IBD has been associated with its “non-specific anti-inflammatory” activity (Stenson & Korznik (2003) supra) with the suggestion that sulfasalazine does not exhibit an antimycobacterial effect in Crohn's disease (Van Caekenberghe (1989) J. Lab. Clin. Med. 114:63-5).

The diagnosis and treatment of diseases and conditions herein posited to be the result of a MAP infection are needed. The present invention meets this need in the art by providing the detection and treatment of MAP in a variety of diseases and conditions including, e.g., ulcerative colitis, irritable bowel syndrome, Crohn's Disease, rheumatoid arthritis, Multiple Sclerosis and Alzheimer's Disease.

### SUMMARY OF THE INVENTION

The present invention is a method for inhibiting the proliferation of Mycobacterium avium subspecies paratuberculosis. This method involves contacting a M. avium subspecies paratuberculosis (MAP) with an effective amount of at least two anti-MAP agents, or a prodrug or metabolite thereof, selected from the group consisting of a folate antagonist, a purine inhibitor, a thymidylate synthase inhibitor, an antibiotic, an immunosuppressant and a thalidomide, so that proliferation of the MAP is inhibited. In particular embodiments, the combination of anti-MAP agents is co-administered with a salicylic acid. In alternative embodiments, MAP proliferation is inhibited by contacting a MAP with an effective amount of a salicylic acid alone, or in combination with a folate antagonist.

The present invention also embraces a method for treating a MAP infection by administering to a patient having or at risk of having a MAP infection an effective amount of at least two anti-MAP agents, or a prodrug or metabolite thereof, selected from the group consisting of a folate antagonist, a purine inhibitor, a thymidylate synthase inhibitor, an antibiotic, an immunosuppressant and a thalidomide. In particular embodiments, the combination of anti-MAP agents is co-administered with a salicylic acid. In alternative embodiments, a MAP infection is treated by administering an effective amount of a salicylic acid alone, or in combination with a folate antagonist.

The present invention is also a pharmaceutical composition composed of at least two anti-MAP agents, or a prodrug or metabolite thereof, selected from the group consisting of a folate antagonist, a purine inhibitor, a thymidylate synthase inhibitor, an antibiotic, an immunosuppressant and a thalidomide in admixture with a pharmaceutically acceptable carrier. A pharmaceutical composition composed of a salicylic acid and a folate antagonist in admixture with a pharmaceutically acceptable carrier is also provided.

The present invention further embraces a method for diagnosing Multiple Sclerosis or Alzheimer's Disease. The method involves detecting the presence of MAP nucleic acid molecules in a sample from a subject having or suspected of having Multiple Sclerosis or Alzheimer's Disease, wherein the presence of MAP nucleic acid molecule in the sample is indicative of Multiple Sclerosis or Alzheimer's Disease. In particular embodiments, the diagnostic method includes the prestep of in vitro culturing of MAP from the sample.

A method for determining the antibiotic susceptibility of MAP isolated from a subject is also provided. This method involves growing MAP from a sample under suitable conditions to obtain the cell wall containing form of MAP and contacting the MAP with a plurality of antibiotics to determine the antibiotic susceptibility of the MAP.

The present invention further relates to a method for inhibiting the proliferation of MAP by contacting MAP with an effective amount of an immunosuppressant so that proliferation of the MAP is inhibited. In particular embodiments, the immunosuppressant is a cyclosporine or a macrolide immunosuppressant

### BRIEF DESCRIPTION OF THE DRAWINGS

FIG. 1 is a bar graph showing the cumulative GI data evaluating MAP ATCC 19698. Each drug dilution was studied in duplicate. Error bars are SD. There are three control inoculations, labeled on the abscissa as “0, 00 & 000. The left hand “0” had an equal number of CFUs as in each drug studied. “00”=10 & “000”=100-fold dilutions. In each control the maximum concentration of diluent used for each agent (methanol for Clarithromycin, water for ampicillin and NaOH for methotrexate and 6-MP) was added.

FIG. 2 shows graphs of the growth inhibition of MAP ATCC 19698 (FIG. 2A), MAP Dominic (FIG. 2B), M. avium ATCC 25291 (FIG. 2C) and M. avium 101 (FIG. 2C) in the presence of inhibitors. “0” is diluent control with an equal CFU inoculum and “00” is a 1:10 dilution of the water control inoculum. Drug concentrations are indicated on the abscissa. For each isolate, drug dose was studied in singlicate.

FIG. 3 shows graphs of the growth inhibition of MAP ATCC 19698 (FIG. 3A) and M. avium 101 (FIG. 3B) in the presence of salicylic acids, 5-ASA and p-aminosalicylic acid (PAS) and folate antagonist sulfapyridine (Sulfapyr). The difference in the “baseline” for p-aminosalicylic acid is because of the diluent used to solubilize it this compound.

### DETAILED DESCRIPTION OF THE INVENTION

Analogous to lepromatous leprosy and tuberculoid leprosy, it is now posited that Multiple Sclerosis and perforating Crohn's disease are the “acute” forms of a Mycobacterium avium subspecies paratuberculosis (MAP) infection, whereas Alzheimer's Disease and obstructive Crohn's or ulcerative colitis are the chronic forms of a MAP infection. It is further posited that a causative relationship between MAP and diseases such as IBD and Multiple Sclerosis have been missed because it has not been appreciated that standard treatment regimes, whose mechanisms of actions are unknown or speculated upon, are in fact effective because they are treating a MAP infection. The panoply of medications that are used to treat diseases such as IBD and Multiple Sclerosis can be divided into two groups; one that treats a MAP infection and the other that treats the inflammatory condition that is consequent to the primary infection.

An example of a medication which treats the primary MAP infection that causes IBD is salfasalazine, i.e., sulfapyridine conjugated to what is presumed to be an anti-inflammatory agent 5-ASA. Upon cleavage by bacterial azo reductase, the 5-ASA moiety is liberated from sulfapyridine in the colon. Examples of the second group of medications which treat the secondary, inflammatory component of the infection include the corticosteroids (e.g., cortisol and budesonide) and the anti-TNF-alpha monoclonal antibody infliximab. Such actual anti-inflammatory medications for treating the secondary effect of the primary infection, are not embraced by the primary therapeutic agents disclosed herein.

The present invention embraces compositions and methods for inhibiting the proliferation of MAP and treating or ameliorating a MAP infection. Specifically, the present invention provides novel anti-MAP agents and combinations of anti-MAP agents for suppressing the growth of MAP thereby treating diseases or conditions associated with MAP (i.e., MAP is the etiological agent of the disease or condition).

Compounds disclosed herein are particularly useful for their bactericidal or bacteriostatic activity, i.e., the degree of efficacy of an antibacterial agent. Antimicrobial compositions can effect two kinds of microbial cell damage. The first is a lethal, irreversible action resulting in complete microbial cell destruction or incapacitation as observed by a decrease in cell proliferation and/or the number of viable cells. The second type of cell damage is reversible, such that if the organism is rendered free of the agent, it can again multiply. The former is termed bactericidal and the later, bacteriostatic.

MAP has a long division time and, akin to the treatment of Mycobacterium tuberculosis and M. leprae, antimicrobial therapy for the treatment of MAP has to be carried out for a long period of time with a risk of the generating resistant strains. Thus, while some embodiments embrace the use of one anti-MAP agent as the primary therapeutic agent, other embodiments embrace the simultaneous use of multiple anti-MAP agents to prevent the emergence of resistant mycobacterium in the treatment of a MAP infection. Thus, in particular embodiments, the present invention provides the combination of at least two, three, four, five, six, seven or more anti-MAP agents disclosed herein.

When employing a single anti-MAP agent, certain embodiments embrace the use of a salicylic acid or prodrug thereof as the primary or sole therapeutic to inhibit the proliferation of MAP. Salicylic acids of use in accordance with the this invention include, 5-ASA and derivatives thereof, e.g., 4-ASA (also known as p-aminosalicylic acid), NCI60-676931, CAS 69727-10-2, 3-aminosalicylic acid, CAS 4136-97-4, CAS 2374-03-0, acetylsalicylic acid, 5-bromo-2-acetylsalicylic acid, N-acetyl-5-aminosalicylic acid; prodrugs thereof, e.g., 5-aminosalicyl-L-aspartic acid and 5-aminosalicyl-L-glutamic acid (see Jung, et al. (2001) J. Pharm. Sci. 90(11):1767-75); and pharmaceutically acceptable salts thereof, e.g., potassium acetylsalicyclic acid (see, U.S. Pat. No. RE38,576). As used in the context of the present invention, a prodrug is an active compound that is modified by, e.g., conjugation with an inactive compound, to facilitate delivery or decrease toxicity of the active compound until it reaches its target site where it undergoes biotransformation via a metabolic process before exhibiting its pharmacological effects. Thus, in one embodiment, the salicylic acid of the instant invention is an aminosalicylic acid or prodrug thereof. In another embodiment, the salicylic acid is 5-ASA or a prodrug thereof.

In certain embodiments, the present invention embraces the administration of a folate antagonist either alone or in combination with a salicylic acid, wherein said salicylic acid and folate antagonist are not conjugated at the time of administration. In other embodiments, the salicylic acid and folate antagonist are not conjugated at the site of action (i.e., at the site of the bacterial infection).

As used in the context of the present invention, a folate antagonist is an agent that inhibits one or more enzymes of the folate biosynthetic pathway. For the purposes of this invention, the term folate antagonist expressly includes folate antagonist prodrugs and folate antagonist metabolites. A metabolite is a product of a parent compound which undergoes biotransformation via a metabolic process; however, the parent compound may also exhibit a pharmacological effect. Sulfonamides are a well-known group of folate antagonists that are mostly derivatives of sulfanilamide (p-aminobenzenesulfonamide). Organisms which synthesize their own folic acid and which cannot use an exogenous supply of the vitamin are sensitive to sulfonamides. This is a result of the ability of sulfonamides to act as structural analogs of p-aminobenzoic acid (PABA) and competitively inhibit the incorporation of PABA during folic acid synthesis. For example, methotrexate is a folate antagonist that competitively and reversibly inhibits DHFR and has been used in the treatment Crohn's disease (Feagan, et al. (2000) N. Engl. J. Med. 342:1627-32). Similarly, it is posited that sulfapyridine, a sulfonamide cleavage product of the IBD therapeutic salfasalazine, also functions as a folate antagonist which inhibits the proliferation of MAP.

There are many sulfonamide drugs that differ in their clinical properties and toxicities. Most are derivatives bearing substituents at the nitrogen of the sulfonamide group (i.e., NH₂C₆H₄SO₂NHR, wherein R represents the substituent). Substitution at the p-amino group normally results in loss of antibacterial activity. However, such derivatives are often hydrolyzed in vivo to an active form and can therefore be administered in an inactive form. For example, p-N-succinylsulfathiazole and phthalylsulfathiazole are inactive but are hydrolyzed in the lower intestine to release the active component sulfathiazole.

Exemplary folate antagonists include sulfonamides such as sulfanilamide; sulfaphenazole; sulfadiazine; sulfamethizole; sulfisoxazole; sulfamethoxazole (4-amino-N-(5-methyl-1,3,4-thiadiazol-2-yl)benzenesulfonamide); sulfapyridine; sulfadimethoxine (4-amino-N-(2,6-dimethoxy-4-pyrimidinyl)benzenesulfonamide); sulfamonomethoxine; sulfadimidine; sulfamethazine; sulfacetamide (N-[4-aminophenyl)sulfonyl]-acetamide); sulfathiazole (4-azino-N-2-thiaamide); sulfaguanidine (4-amino-N-(aminoiminomethyl)benzenesulfonamide).

Additional folate antagonists of use in the instant compositions and methods include, but are not limited to, aminopterin, methotrexate, trimetrexate, LY231514, and 5-phosphoribosylglycinamide formyltransferase (GARFT) inhibitors such as lometrexol. Moreover, pyridopyrimidines that inhibit the activity of dihydrofolate reductase (DHFR) from Pneumocystis carinii, Toxoplasma gondii, and Mycobacterium avium (Chan, et al. (2005) J. Med. Chem. 48:4420-31), are also embraced by the present invention. In one embodiment, the folate antagonist is a sulfonamide. In other embodiment, the folate antagonist is sulfapyridine.

As indicated, some embodiments embrace a salicylic acid used alone or in combination with a folate antagonist. However, other embodiments of this invention provide the use of one, two, three, four or more anti-MAP agents selected from the group consisting of a folate antagonist, a purine inhibitor, a thymidylate synthase inhibitor, an antibiotic, an immunosuppressant and a thalidomide. In particular embodiments, at least two anti-MAP agents are employed.

During the de novo biosynthesis of purines and thymine, methyl groups are donated by tetrahydrofolate, which is regenerated from the dihydrofolate product of thymidylate synthase by the action of DHFR. Thus, folate antagonists of DHFR indirectly inhibit the biosynthesis of purines and thymine. The 6-mercaptopurine metabolite of azathioprine, an IBD therapeutic, is a purine analogue that acts by directly interfering with purine synthesis. Accordingly, the use of agents which indirectly interfere with purine biosynthesis are included within the scope of anti-MAP agents of this invention. Such inhibitors include purine inhibitors such as 6-mercaptopurine, azathioprine, 6-thioguanine, and 6-methylmercaptopurine riboside (see U.S. Pat. No. 6,355,623) and their prodrugs and metabolites; and thymidylate synthase inhibitors such as raltitrexed (Tomudex), nolatrexed, LY231514, ZD9331, and 5-fluorouracil and its prodrugs, 5′-deoxy-5-fluorouridine and ftorafur, which are metabolized to fluorodeoxyuridine monophosphate. In particular embodiments, the purine inhibitor 6-mercaptopurine is used as an anti-MAP agent.

The structure of thalidomide is similar to that of the DNA purine bases adenine (A) and guanine (G).

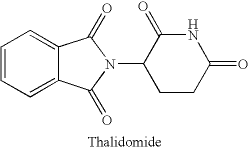

In solution, thalidomide binds more readily to guanine than to adenine, and has almost no affinity for the other nucleotides, cytosine (C) and thymine (T). It is noteworthy that the progenitor molecule of these two purine double ringed nucleotides is the double ringed dihydrofolate that is converted to folic acid prior to becoming the purine nucleotides. There is thus a parallelism between this and the posited action of methotrexate, a dihydrofolate reductase inhibitor which interferes with the DNA replication of the infecting mycobacterium. As thalidomide can intercalate into DNA at G-rich sites (Stephens, et al. (2000) Biochem Pharmacol. 59(12):1489-99) and the 1H-isoindole-1,3(2H)-dione thalidomide metabolite may bind to replication/transcription machinery given the structural similarity to purines (Scheme 1), it is contemplated that thalidomide, or active metabolites thereof, exerts its anti-mycobacterial effect by disrupting prokaryotic DNA replication.

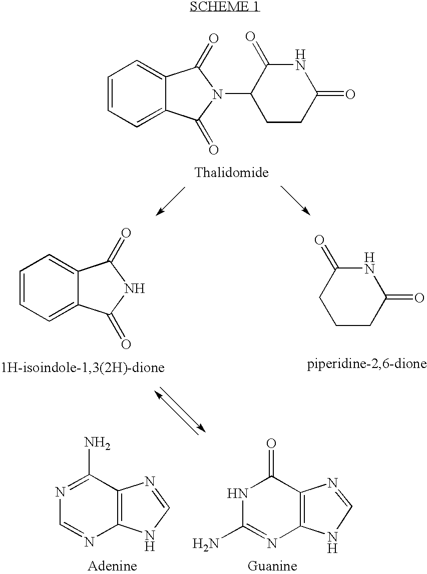

In this regard, the piperidine metabolite of thalidomide is the basis for a well-known class of antimicrobial agents, namely glutarimides such as streptimidone, streptovitacins and inactone (see, e.g., Kim, et al. (1999) J. Agric. Food Chem. 47(8):3372-80; Sugawara, et al. (1992) J. Antibiot. (Tokyo) 45(9):1433-41; Sonoda, et al. (1991) J. Antibiot (Tokyo) 44(2):160-3), and it has now been demonstrated that 1-hydroxy-2,6-piperidinedione (HPD) inhibits the growth of MAP. Thus, certain embodiments of the present invention embrace the use of either the isoindole metabolite or piperidine metabolite alone or, as has been indicated for the breakdown products of sulfasalazine (i.e., sulfapyridine and 5-ASA), in combination to act synergistically in the inhibition of the proliferation of multiple Mycobacterium species and subspecies. Such synergy between the two metabolites of thalidomide can be assessed in vivo in a clinical study, e.g., in either animals or humans, where the intact thalidomide molecule, or its components individually and in combination are prospectively compared.

Moreover, it is posited that the metabolites of thalidomide analogs such as lenalidomide and CC-4047, differing by substituents of the isoindole, will be more potent antimicrobial agents than the metabolites of thalidomide itself as these analogs are at least 2000 times more potent than thalidomide in inhibiting the TNF-α (Teo, et al. (2005) Drug Discovery Today 10:107-114).

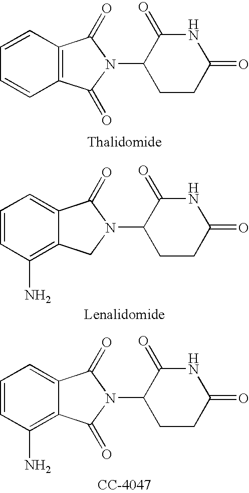

Thus, in accordance with a particular embodiment of the present invention, a thalidomide is used in the composition and methods of the present invention. As used in the context of the present invention, the term thalidomide is intended to include, but is not limited to, thalidomide, a derivative or analog of thalidomide, or a pharmaceutically acceptable prodrug (i.e., a compound which is metabolized to produce thalidomide or analogues or biologically active salt forms thereof), a metabolite, salt, solvate, hydrate, or clathrate thereof. Thalidomide contains a chiral center, and is commercially available as a racemate. The methods and compositions of the invention therefore encompass the use of racemic thalidomide as well as optically pure enantiomers of thalidomide. Optically pure enantiomers of thalidomide can be prepared by methods well-known in the art. These include, but are not limited to, resolution of chiral salts, asymmetric synthesis, or chiral chromatography. It is further contemplated that pharmaceutically acceptable prodrugs, salts, solvate, clathrates and derivatives of thalidomide be used in the methods and compositions of the invention. Examples of derivatives of thalidomide that can be used in the methods and compositions of the invention include, but are not limited to, taglutimide, supidimide, and those disclosed in WO 94/20085. Other derivatives of thalidomide encompassed by this invention include, but are not limited to, 6-alkyl-2-[3′-nitrophthalimido]-glutarimide, 6-alkyl-2-[4′-nitrophthalimido]-glutarimide, 6-alkyl-3-phenylglutarimides, CPS11, CPS49, IMiD3, lenalidomide and CC-4047. See, e.g., De and Pal (1975) J. Pharm. Sci. 64(2):262-266 and U.S. Pat. No. 6,235,756. Moreover, 5-OH-thalidomide and 5′-OH-thalidomide metabolites, which exhibit biological activity in vitro (Price, et al. (2002) Ther. Drug Monit. 24(1):104-10), are also contemplated for use in the instant methods, as are 4-OH-thalidomide and N—OH-thalidomide. In some embodiments, the thalidomide itself exhibits activity, whereas in other embodiments, one or more metabolites of the thalidomide exhibit activity (see Scheme 1).

With respect to embodiments drawn to metabolites of thalidomide, e.g., the isoindole metabolite or piperidine metabolite, the present invention also embraces a derivative or analog of a thalidomide metabolite, or a pharmaceutically acceptable prodrug, salt, solvate, hydrate, or clathrate thereof. By way of illustration, piperidine-2,6-dione analogues of particular use include, but are not limited to, aminoglutethimide, p-nitro-glutethimide, p-nitro-5-aminoglutethimide, cyclohexy-laminoglutethimide, phenglutarimide, acetylamino-glutethimide, glutethimide, pyridoglutethimide, 3-phenylacetyl-amino-2,6-piperidinedione (antineoplaston A-10), and thioglutethimide.

Another embodiment of the present invention includes the use of an anti-MAP antibiotic, or a prodrug or metabolites thereof. For example, macrolide antibiotics are known for use in the treatment of MAP. Macrolide antibiotics of the invention include clarithromycin and azithromycin. Other anti-MAP antibiotics of use in conjunction with the anti-MAP agents identified herein include, but are not limited to, rifabutin, amikacin, cyprofloxicillin, clofazimine, rifampicin, azithromycin, roxithromycin, ethambutol, ofloxacin, ciprofloxacin, metroniadiazole and oxazolidinone.

Having demonstrated the anti-MAP activity of immunosuppressants, particular embodiments of this invention further embrace the use of an immunosuppressant, either alone or in combination with one or more additional anti-MAP agents for inhibiting the proliferation of MAP and treating a MAP infection. An immunosuppressant is defined herein as an agent that can suppress or prevent the immune response. In particular embodiments of the invention, the immunosuppressant employed is a cyclosporin or macrolide immunosuppressant. A cyclosporin (also referred to as cyclosporine) is defined herein as a cyclic nonribosomal peptide containing one or more D-amino acids. Cyclosporins of use in accordance with the present invention include, but are not limited to Cyclosporine A, Cyclosporin G (OG37-325), and O-hydroxyethyl-D(Ser)₈-cyclosporine (SDZ IMM 125).

A macrolide immunosuppressant is defined herein as a class of compounds sharing a macrolide-like structure composed of a large macrocyclic lactone ring to which one or more deoxy sugars, usually cladinose and desosamine, may be attached. In general, the lactone rings of macrolide immunosuppressants are 14, 15 or 16-membered. Exemplary macrolide immunosuppressants of the invention include, but are not limited to sirolimus (rapamycin), tacrolimus (FK506), pimecrolimus, and everolimus.

As indicated herein, it is posited that the metabolic products of sulfasalazine, namely sulfapyridine and 5-ASA, both exhibit antimicrobial activity. Accordingly, particular embodiments further embrace combining the one, two, three or more anti-MAP agents selected from the group consisting of a folate antagonist, a purine inhibitor, a thymidylate synthase inhibitor, an antibiotic, an immunosuppressant and a thalidomide with a salicylic acid.

When used therapeutically, the one or more anti-MAP agents of the present invention are usually administered in the form of an anti-MAP pharmaceutical composition, wherein the agents are in admixture with a pharmaceutically acceptable carrier. Such a pharmaceutical composition can be prepared in any manner well-known in the pharmaceutical art. See, e.g., Remington: The Science and Practice of Pharmacy, Alfonso R. Gennaro, editor, 20th ed. Lippincott Williams & Wilkins: Philadelphia, Pa., 2000. For example, in making the pharmaceutical composition of the invention, the anti-MAP agents are usually mixed with a carrier, diluted by a carrier or enclosed within such a carrier which can be in the form of a capsule, sachet, paper or other container. When the carrier serves as a diluent, it can be a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In this regard, the anti-MAP composition can be administered by a variety of routes including oral, rectal, transdermal, subcutaneous, intravenous, intramuscular, intranasal or locally, e.g., intraspinal, intracerebral, and the like.

Suitable carriers for use in accordance with the instant invention include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methylcellulose. The formulations can additionally include lubricating agents such as talc, magnesium stearate, and mineral oil; wetting agents; emulsifying and suspending agents; preserving agents such as methyl- and propylhydroxybenzoates; sweetening agents; and flavoring agents. The pharmaceutical compositions of the invention can be formulated so as to provide quick, sustained or delayed release of the active ingredient after administration to the subject by employing procedures known in the art.

In particular embodiments, the pharmaceutical composition is enterically coated. For example, tablets, capsules or pills can be coated or otherwise compounded to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac and cellulose acetate.

In treating a MAP infection of the CNS it may be desirable that the one or more anti-MAP agents be attached to a delivery moiety or entrapped within a drug carrier (e.g., liposomes and nanoparticles), which facilitates delivery across the blood-brain barrier. See, e.g., Koziara, et al. (2006) J. Nanosci. Nanotechnol. 6(9-10):2712-35; and de Boer and Gaillard (Sep. 8, 2006) Annu Rev Pharmacol Toxicol. [Epub ahead of print] PMID: 16961459.

For the purposes of the present invention, the anti-MAP agents of the invention can be administered simultaneously or sequentially (one immediately following the other) with the proviso that when a folate antagonist is used in combination with a salicylic acid, the folate antagonist and salicylic acid are not conjugated.

As discussed herein, it is believed that the causative relationship between MAP and diseases such as IBD, Multiple Sclerosis and Alzheimer's Disease, as well as other infectious agents in other inflammatory diseases, has been missed because it has not been appreciated that standard inflammatory disease treatment regimes are actually treating the infectious entity. For example, it is believed that the mechanism of action of the individual components of sulfapyridine is to retard the growth of MAP in IBD. Likewise, it is contemplated that the anti-metabolites disclosed herein (i.e., folate antagonists such as methotrexate, purine inhibitors such as 6-mercaptopurine and thymidylate synthase inhibitors) exhibit antibiotic activity in addition to their anti-neoplastic activity. It is believed that anti-metabolites interfere with common metabolic pathways in both eukaryotes and prokaryotes; however because prokaryotes are more sensitive to these agents, much lower doses can be achieved in the treatment of inflammatory diseases resulting from bacterial infections as compared to doses employed in the treatment of human malignancies.

For example, methotrexate and 6-mercaptopurine are used in the therapy of malignancies as well as inflammatory diseases. Remarkably, these agents are employed at different doses; an anti-neoplastic dose and an anti-inflammatory dose.

The anti-metabolite 6-mercaptopurine and its metabolite azathioprine are used to treat two generic types of disease, malignancies and inflammatory diseases (Calabresi & Parks (1977) In: Goodman L S, Gilman A, eds. The Pharmacological Basis of Therapeutics. Fifth ed. New York: Macmillan, pg. 1254-1307). The mechanism of action is “to inhibit nucleotide biosynthesis and purine nucleotide interconversion.” (Berardi (1996) In: Herfindal E T, Gourley D R, eds. Textbook of Therapeutics. Drugs and Disease Management. Baltimore: Williams and Wilkins, pg. 483-502).

“High dose” treatment is used in reticuloendothelial malignancies such as acute leukemia. In such cases the starting dose is 100-200 mg/day 6-mercaptopurine titrated to a maintenance dose of 50-100 mg/day (Calabresi & Parks (1977) supra; Calabresi & Chabner (1990) In: S. G L, Gilman A, Rall T, Nies A S, Taylor P, eds. The Pharmacological Basis of Therapeutics. 8 ed. New York: Pergamon Press, pg. 1202-1208). The cumulative dose that is used, until bone marrow depression occurs, in non-hematological malignancies is ˜45 mg/kg (Range 18-106 mg/kg). Thus, a 70 kg man might receive an acute cumulative dose of 6-mercaptopurine of >7 grams.

In contrast, “low dose” long-term 6-mercaptopurine is used in the therapy of inflammatory diseases such as IBD and rheumatoid arthritis at a dose that is at ˜≦25% of that used in malignancies. The typical starting dose is 0.5 to 1.5 mg/kg/day 6-mercaptopurine with a usual maximum of ≦50 mg/day (Bernstein, et al. (1994) Dig. Dis. Sci. 39(8):1638-41). At this dosage, minimal hematological toxicity is caused.

Similarly, methotrexate is used in “high” and “low” doses for treatment of different diseases. For example, the high dose is used for malignancies such as non-Hodgkin's lymphoma (Urba, et al. (1990) J. Natl. Cancer Inst. Monogr. 10:29-37). In combination with other anti-neoplastic agents, the dose of methotrexate is 120 mg/m² over 8 days as an oral dosage, and 1500-5000 mg M² by IV infusion over 2-24 hours (Findley & Fortner (1996) In: Herfindal E T, Gourley D R, eds. Textbook of Therapeutics: Drugs and Disease Management. Sixth ed. Baltimore Md.: Williams & Wilkins, pg. 1509-1513).

In contrast, “low” dose methotrexate is used for inflammatory diseases such as such as IBD, rheumatoid arthritis and psoriasis. In IBD, (Crohn's and ulcerative colitis) a weekly injection of 25 mg improves symptoms and decreases the required dose of corticosteroids (Feagan, et al. (1995) N. Engl. J. Med. 332(5):292-7). In the treatment of rheumatoid arthritis the dose of methotrexate is 7.5 to 15 mg orally or by IM injection weekly (Weinblatt, et al. (1992) Arthritis Rheum. 35(2):129-37; Weinblatt, et al. (1998) J. Rheumatol. 25(2):238-42). Similarly, in the therapy of psoriasis, the dose of methotrexate is 5 mg to 7.5 mg weekly.

The effectiveness of these “low” dose 6-mercaptopurine and methotrexate regimes in the treatment of inflammatory diseases is herein posited to be as a result of antibiotic activity which decreases bacterial infectious burden, with a decrease in pro-inflammatory cytokines as a secondary phenomenon.

Similarly, there are disparate doses of thalidomide that are used to treat malignant, infectious and “inflammatory” diseases. When used to treat malignancies, (such as the reticuloendothelial malignancy Multiple Myeloma) the initial dose of thalidomide is 200 mg/day increasing to 1200 mg/day (Singhal, et al. (1999) N. Engl. J. Med. 341(21):1565-71). In contrast, a lower dose is used to treat the acknowledged infectious disease erythema nodosum leprosum (ENL,) that is caused by M. leprae. The initial dose of thalidomide for ENL is 200 mg/day. It is increased to 400 mg/day, but the maintenance dose is 50 mg/day to 200 mg/day (Iyer, et al. (1971) Bull. World Health Organ. 45(6):719-32). Thus, the maintenance dose of thalidomide for a mycobacterial infectious disease may be one sixteenth of that used for reticuloendothelial malignancies. The dose of thalidomide that is used in the therapy of Crohn's disease (herein posited to be primarily infectious and due to M. paratuberculosis rather than primarily “inflammatory”) is similar to that used in ENL the other mycobacterial disease discussed, and is one-eighth the dose used to treat reticuloendothelial malignancies. In refractory Crohn's disease, the starting dose of thalidomide is 200 mg/day, decreasing to maintenance of 100 mg/day to 200 mg/day (Ehrenpreis, et al. (1999) Gastroenterology 117(6):1271-7).

Likewise, “high” and “low” doses of immunosuppressants are used depending on whether the immunosuppressant is being administered to reduce organ transplant rejection or in the treatment of “inflammatory” diseases. For example, the targeted trough level of cyclosporine A in transplant rejection is 100 ng/ml to 400 ng/ml (Wong (2001) Clin. Chim. Acta 313(1-2):241-53; van der Pijl (1996) Transplantation; 62(4):456-62; Lindholm & Kahan (1993) Clin. Pharmacol. Ther. 54(2):205-18), whereas levels in the range of 70 ng/ml to 200 ng/ml are recommended in the treatment of Crohn's disease. “High” dose targeted trough levels of tacrolimus, in the range of 5 ng/mL to 20 ng/mL (Wong (2001) supra), are used, e.g., in liver transplantation (Sugawara, et al. (2001) Clin. Transplant. 16(2):102-6), whereas “low” targeted trough levels of 4-8 ng/ml, or 5-10 ng/ml are recommended in the treatment of inflammatory bowel disease (Baumgart, et al. (2006) Am. J. Gastroenterol. 101(5):1048-56; de Oca, et al. (2003) Rev. Esp. Enferm. Dig. 95(7):465-70, 459-64).

Accordingly, an effective amount of each anti-MAP agent can be routinely determined by the skilled physician, in the light of the teachings herein and the relevant circumstances, including the condition to be treated, the chosen route of administration, the actual compound administered, the age, weight, and response of the individual subject, the severity of the subject's symptoms, and the like. As used herein, a subject is intended to include humans; however, the treatment of livestock, zoological, and companion animals is also contemplated.

In general, treatment of a MAP infection is achieved by administering to a subject having or at risk of having a MAP infection an effective amount of an anti-MAP composition of the present invention so that proliferation of MAP or MAP cell viability is decreased as compared to proliferation or cell viability prior to commencement of treatment. In so far as the MAP infection being treated is associated with an inflammatory bowel disease (IBD), Multiple Sclerosis or Alzheimer's Disease, treatment will also improve or ameliorate at least one sign or symptom of the IBD, Multiple Sclerosis or Alzheimer's Disease or the maintenance of disease remission.

As used routinely in the art, IBD is used herein to refer to both ulcerative colitis and Crohn's disease. Ulcerative colitis causes inflammation of the lining of the large intestine or rectum, whereas Crohn's disease causes inflammation of the lining and wall of the large and/or small intestine. Accordingly, ulcerative colitis subjects benefiting from the administration of one or more anti-MAP agents of the invention may have less diarrhea, fewer abdominal cramps, or a decrease in rectal bleeding as compared to prior to commencement of treatment. Similarly, Crohn's disease subjects benefiting from the administration of one or more anti-MAP agents of the invention may have a decrease in abdominal pain, diarrhea, and rectal bleeding as compared to prior to commencement of treatment.

Likewise, subjects having or at risk of having Multiple Sclerosis benefit from the anti-MAP agents by an amelioration or elimination of one or more signs or symptoms including, but not limited to, blurred vision, muscle weakness or paralysis, numbness, loss of coordination, and the like; whereas subjects having or at risk of having Alzheimer's Disease benefit by an amelioration or elimination of one or more signs including, but not limited to, memory loss, problems with language, disorientation of time and place, and the like.

As used herein, a subject at risk of having a MAP infection is intended to include a subject in remission of an IBD, Multiple Sclerosis or Alzheimer's Disease, or a subject having a genetic defect that is know to be associated with a predisposition for contracting IBD, e.g., subjects with a defect in NOD2 (Hugot, et al. (2001) Nature 411(6837):599-603; Ogura, et al. (2001) Nature 411(6837):603-606) or NRAMP1 (Kojima, et al. (2001) Tissue Antigens 58(6):379-84), wherein it is herein posited that these defects place the subject in an immunocompromised state thereby impeding the ability of the subject to combat the MAP infection. Subjects having or at risk of having a MAP infection include subjects with irritable bowel syndrome (IBS) or an inflammatory bowel disease identified by IBD diagnostic methods such as routine flexible sigmoidoscopy or colonoscopy examination, barium enema, or small bowel X-ray, capsule endoscopy as well as subjects identified by routine screening methods for Multiple Sclerosis or Alzheimer's Disease.

Optionally, a subject having or at risk of having a MAP infection can also be diagnosed by detecting the presence of MAP antigens (e.g., ELISA) or MAP nucleic acids (e.g., DNA, RNA such as an rRNA or mRNA specific to MAP) in a sample from the subject. As used herein, a sample can be blood, a stool sample, cerebrospinal fluid or alternatively a biopsy sample, e.g., lesioned central nervous tissue or a biopsy obtained in endoscopy or a surgically resected specimen.

In man, as opposed to ruminants, MAP is found in vivo in the cell wall-deficient form. As such, propagation and detection of MAP based solely on conventional growth plate-based assays is difficult. Thus, some embodiments of the present invention embrace directly detecting the MAP nucleic acid molecule without manipulation of the sample, while other embodiments embrace isolating the nucleic acid molecule and/or isolation of the MAP bacterium by in vitro culturing.

In vitro culturing of MAP bacterium from a sample (e.g., lesioned tissue or cerebrospinal fluid) involves placing the sample on an appropriate growth medium under suitable conditions to obtain the cell wall containing form of MAP. Such suitable conditions are known in the art and include the commercially available Mycobacteria Growth Indicator Tube (MIGT) system (Becton-Dickerson) which is an automated system containing a rich growth medium for growing MAP. To obtain a sufficient amount of MAP, the MAP can be grown for one, two, three or more months.

For the purposes of the present invention, the MAP nucleic acid molecule can be detected in situ or in vitro. The in situ detection of MAP nucleic acid molecules is well-known in the art. See, e.g., Hulten, et al. (2001) Am. J. Gastroenterology 96:1529). In vitro detection generally involves the isolation of all (e.g., RNA and DNA) or a portion (i.e., RNA or DNA) of the total nucleic acids from a sample. Such methods are well-known to those of skill in the art. For example, methods of isolation and purification of nucleic acids are described in detail in Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993).

In the detection of MAP-specific RNA molecules, total RNA can be isolated from a given sample using, for example, an acid guanidinium-phenol-chloroform extraction method. See, e.g., Gilberts, et al. (1994) Proc. Natl. Acad. Sci. USA 91:12721-12724. Frequently, it is desirable to amplify the nucleic acid sample prior to detection. One of skill in the art will appreciate that methods of amplifying nucleic acids are well-known in the art. Such suitable amplification methods include, but are not limited to polymerase chain reaction (PCR) (Innis, et al. (1990) PCR Protocols. A guide to Methods and Application. Academic Press, Inc., San Diego), ligase chain reaction (LCR) (see Wu and Wallace (1989) Genomics 4:560; Landegren, et al. (1988) Science 241:1077; Barringer, et al. (1990) Gene 89:117), transcription amplification (Kwoh, et al. (1989) Proc. Natl. Acad. Sci. USA 86:1173), and self-sustained sequence replication (Guatelli, et al. (1990) Proc. Nat. Acad. Sci. USA 87:1874).

In certain embodiments, the sample mRNA is reverse transcribed with a reverse transcriptase and primers such as a collection of random oligonucleotides is used to generate a single-stranded DNA template. The second DNA strand is polymerized using a DNA polymerase. Second strand DNA synthesis can be specific or non-specific, i.e., the second strand can be synthesized with using one or more oligonucleotides which specifically hybridize to a particular MAP nucleic acid molecule. For example, MAP-specific insertion sequence, IS 900, can be amplified using primers 5′-GAA GGG TGT TCG GGG CCG TCG CTT AGG-3′ (SEQ ID NO:1) and 5′-GGC GTT GAG GTC GAT CGC CCA CGT GAC-3′ (SEQ ID NO:2). See, e.g., Mishina, et al. (1996) Proc. Natl. Acad. Sci. USA 93(18):9816 and Millar, et al. (1996) Appl. Env. Microbiol. 62:3446-3452. Successive rounds of transcription from each single cDNA template results in amplified RNA. Methods of in vitro polymerization are well-known to those of skill in the art (see, e.g., Sambrook, supra).

As indicated, detection of MAP nucleic acid molecules (i.e., directly or after amplification) can be achieved using a variety of established methods or combinations of methods including, e.g., northern blot analysis (see, e.g., Sambrook and Russell (2001) supra); oligonucleotide or cDNA fragment hybridization wherein the oligonucleotide or cDNA is configured in an array on a chip or wafer; RNase protection analysis; or RT-PCR, as illustrated herein. Depending on the format, detection can be performed by visual means (e.g., ethidium bromide staining of a gel). Alternatively, detection can involve indirect identification of the product via chemiluminescence, radiolabel or fluorescent label or even via a system using electrical or thermal impulse signals (Bellus (1994) J. Macromol. Sci. Pure Appl. Chem. A311:1355-1376). In accordance with the instant diagnostic method, the presence of a MAP nucleic acid molecule in a sample is indicative of an IBD, rheumatoid arthritis, Multiple Sclerosis or Alzheimer's Disease.

Advantageously, when the diagnostic method of the invention provides the prestep of in vitro culturing of MAP from the sample, the isolated MAP bacterium can be tested for antibiotic susceptibility to effectively select a treatment regime for the subject from which the sample originated. To determine antibiotic susceptibility, the in vitro cultured MAP is contacted with a plurality of antibiotics (e.g., 2, 3, 4, 5, 10, or more). Antibiotic susceptibility testing can be carried out using, e.g., the MIGT or BACTEC systems (both Becton-Dickerson). While the BACTEC system is not highly automated and uses a radionucleotide (¹⁴C), this system is sensitive as it uses a medium that is not very rich and therefore the antibiotics are not idiosyncratically absorbed onto the proteins in the medium.

Based on the insights provided by the instant disclosure, it is contemplated that other anti-MAP agents can be readily screened for and identified for use in the treatment of MAP infections, including MAP infections associated with IBD, Multiple Sclerosis and Alzheimer's Disease.

Moreover, given the therapeutic efficacy of sulfasalazine in the treatment of MAP and rheumatoid arthritis, it is contemplated that rheumatoid arthritis, as well as other diseases such as psoriasis, sarcoidosis, and coronary artery disease (CAD), may have an infectious agent such as MAP, Corynbacterium or other infectious agent, which would be inhibited by the therapeutic agents of the present invention. Accordingly, the anti-MAP compositions of the present invention may also be useful in the treatment of other putative infectious disease including, but are not limited to, rheumatoid arthritis, psoriasis, sarcoidosis, and CAD. Furthermore, the inhibitory activity of sulfapyridine, either alone or in combination with 5-ASA, against M. avium subspecies avium provides a novel therapeutic approach to the treatment of M. avium subspecies avium infections.

The invention is described in greater detail by the following non-limiting examples.

### EXAMPLE 1

### Materials and Methods

Four well-characterized strains of mycobacteria were employed. Two were MAP, a bovine isolate, ATCC 19698 (ATCC, Rockville, Md.) and “Dominic” a human isolate from an individual with Crohn's disease (described in Chiodini, et al. (1986) J. Clin. Microbiol. 24:357-363). The M. Avium ssp avium strains were ATCC 25291 (veterinary source) and M. avium 101 (Bertram, et al. (1986) J. Infect. Dis. 154: 194-195). Primary cultures were performed in Middlebrook 7H9 medium supplemented 9:1 with ADC (both DIFCO, Detroit, Mich.). All media for MAP (liquid and agar plates) were supplemented with 1 μg/ml Mycobactin J (Allied Monitor, Fayette, Mo.)

The detergent TWEEN 80 (recommended to prevent mycobacterial clumping) renders clinically resistant strains of MAP inappropriately susceptible to antimicrobials in cell culture (Bertram, et al. (1986) supra). Accordingly, TWEEN 80 was not used herein. To minimize mycobacterial clumping, one ml of the log phase bacterial culture (˜GI of 500) in 7H12 medium in a BACTEC vial was passaged ≧20 times through a 25 gauge needle (Rastogi, et al. (1992) Antimicrob. Agents Chemother. 36:2843-2846) using a one ml syringe (Becton-Dickerson, Franklin Lakes, N.J.), added to five ml 7H9 medium supplemented with Mycobactin J, vortexed and left standing at ambient temperature for 30 minutes. Subsequently, only the upper three of the six ml were used for inoculations. The number of CFU's inoculated was determined by plating serial dilutions of the inoculum onto 7H10 plates (DIFCO) supplemented for MAP with Mycobactin J (1 μg/ml) and counted when colonies were easily visualized (two to six weeks). At the time of passage, additional aliquots were plated onto blood agar plates to ensure that inocula were not contaminated with non-mycobacterial organisms.

To confirm the identity of the species studied, DNA was obtained from the isolates (High Pure Template Prep. Kit; Roche Diagnostics, Indianapolis, Ind.), PCR amplified using primers for IS 900 (MAP) (Kunze, et al. (1991) Mol. Microbiol. 5:2265-2272) and IS 901/2 (Kunze, et al. (1992) J. Clin. Microbiol. 30:2366-2372) (M. avium & specific subspecies including silveticum) as described (Mishina, et al. (1996) Proc. Natl. Acad. Sci. USA 93:9816-9820). Amplicon sizes were determined using 1% agarose gel electrophoresis. DNA sequence analysis was performed commercially, (Genewiz, North Brunswick, N.J.) with sequence comparison performed using BLAST (NLM).

The positive antibiotic control clarithromycin was dissolved in methanol. The negative control antibiotic was ampicillin (Sigma, St Louis, Mo.) which was dissolved in water. Methotrexate and 6-mercaptopurine (both Sigma) were dissolved in NaOH at a maximal final concentration of 50 mM (SIGMA). Control inocula were performed using the maximum concentration of each diluent. Agents were tested in serial dilutions from a minimum of 0.05 μg/ml to a maximum of 64 μg/ml as indicated. Aliquots of chemicals being evaluated were prediluted, stored at −80° C., thawed, used once and discarded.

Data are presented as cumulative growth index (cGI) units ±SD. The effect (or lack thereof) of each agent is presented as the percent decrease in cGI units (% −ΔcGI), compared to the control cGI of that isolate in the diluent (e.g., methanol or NaOH) that was used for the specific chemical being evaluated. cGI data for each experiment is presented until the day prior to any GI reading exceeding the assay limit of “999” (Table 1). Raw data was archived onto EXCEL, and the cumulative results transferred to PRISM (GRAPHPAD, San Diego, Calif.) for final graphing.

### EXAMPLE 2

### Methotrexate and 6-MP Inhibition of MAP Proliferation

Bacterial quantification was performed retrospectively. Accordingly, for experimental reproducibility, bacterial passage and harvesting were performed when the GI was ˜500. Quantification show that the CFU's of the M. avium isolates were approximately 10-fold higher (˜1×10⁷ CFUs/ml), compared to MAP (˜1×10⁶ CFUs/ml) (Table 1). Because of the difference in growth kinetics, M. avium CFU numbers inoculated were ≧10-fold lower than MAP (Table 1).

TABLE 1

| | MAP | | | M. avium | |
|---|---|---|---|---|---|
| | ATCC | ATCC | | ATCC | |
| | 19698 | 19698 | Dominic | 25291 | 101 |
| GI at | 526 | 523 | 548 | 669 | 267 |
| harvest | | | | | |
| Harvested | 8.1 × 10⁵ | 8.2 × 10⁵ | 6.3 × 10⁵ | 9.1 × 10⁶ | 1.2 × 10⁶ |
| # CFU's/ml | | | | | |
| # CFU's | 20,250 | 20,500 | 15,750 | 910 | 120 |
| Inoculated | | | | | |
| Days to | 12 | 13 | 17 | 7 | 5 |
| reach GI | | | | | |
| “999” | | | | | |

Both MAP isolates (ATCC 19698 & Dominic) were Mycobactin J-dependant, were IS 900-positive and had ≧99% homology with the GENBANK accession NC_{—}002944 of MAP. M. avium ATCC 25291 was positive for IS 902 and M. avium 101 was negative for both.

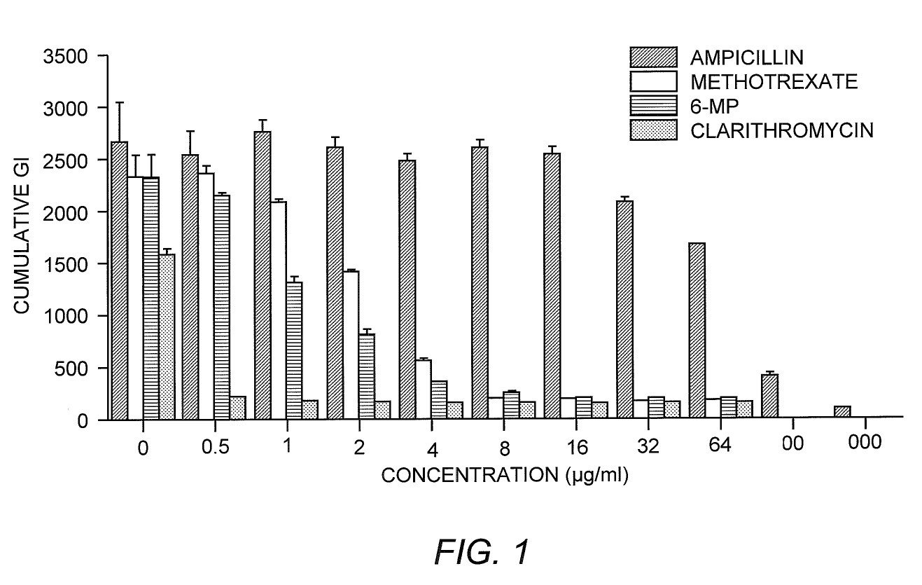

In this study, the positive control antibiotic, clarithromycin exhibited ≧86%−ΔcGI at the lowest concentration evaluated (0.5 μg/ml) (FIG. 1). The negative control antibiotic (the β lactam, ampicillin) had a minimal effect (21% −ΔcGI) at the 32 μg/ml. In contrast, 6-MP had an initial ≧43% −ΔcGI starting at 1 μg/ml increasing to ≧84% −ΔcGI at 4 μg/ml, whereas methotrexate had 40% −ΔcGI inhibition at 2 μg/ml and ≧75% −ΔcGI at 4 μg/ml (FIG. 1).

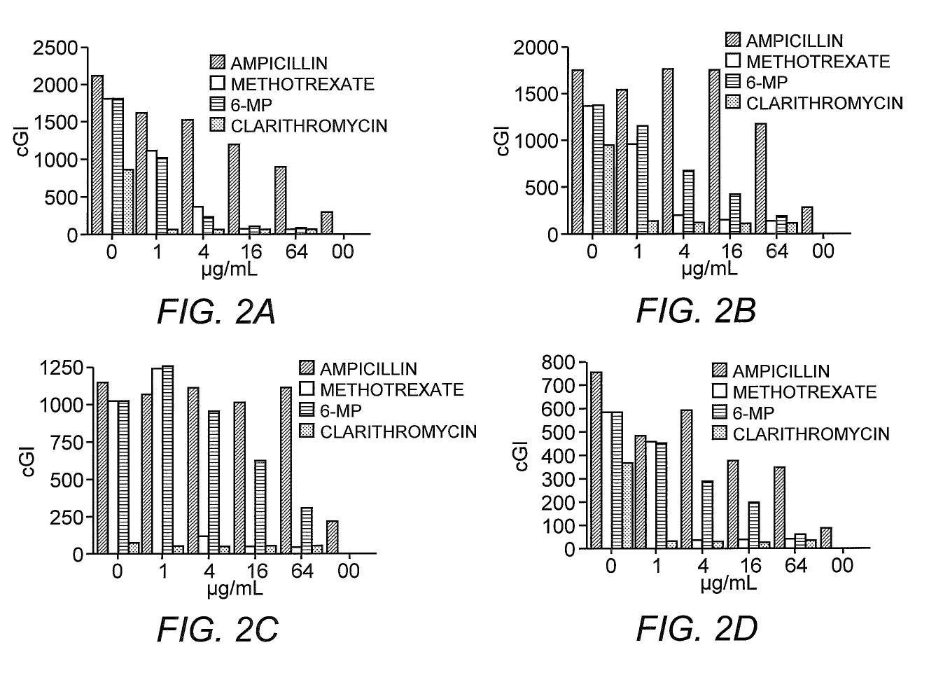

The effect of methotrexate and 6-MP against two MAP (FIGS. 2A and 2B) and two M. avium (FIGS. 2C and 2D) isolates was also evaluated. In these studies, the MAP 19698 results (FIG. 2A) replicated the data presented in FIG. 1 showing ˜80% −ΔcGI inhibition at 4 μg/ml for both 6-MP and clarithromycin. In contrast, MAP Dominic showed less susceptibility to 6-MP (41% −ΔcGI at 4 μg/ml; FIG. 2B) compared to MAP 19698 (84%−ΔcGI at 4 μg/ml). Both M. avium isolates showed less susceptibility to 6-MP than to methotrexate (FIGS. 2C and 2D). The diluent control inoculum for the M. avium ATCC 25291 appeared to exhibit complete growth inhibition (FIG. 2C). However, over the following two days this methanol control entered log phase growth, whereas the vials at every clarithromycin dose continued to show no evidence of growth.

The results of this analysis indicates that both methotrexate and 6-MP inhibit the growth kinetics of MAP. As is conventional with antibiotic susceptibility studies, the present analysis compared agents on an equal weight basis. However, methotrexate (MW 450) is a much larger molecule than 6-MP (MW 170) with a molar ratio of ˜3:1. Thus, in comparison to 6-MP on a molar basis, methotrexate is an even more potent inhibitor of growth than the present data indicate. Moreover, it is expected that combinations of anti-MAP agents will exhibit dose-dependent, growth suppressive activity against MAP which surpass the inhibitory activity of each agent alone. Accordingly, the present invention embraces compositions and methods for inhibiting the proliferation of MAP and treating or ameliorating a MAP infection. Specifically, the present invention provides compositions containing one or more anti-MAP agents including a folate antagonist, a purine inhibitor, a thymidylate synthase inhibitor, an antibiotic, a salicylic acid and a thalidomide, as anti-mycobacterial agents for suppressing the growth of MAP thereby treating diseases or conditions associated with MAP (i.e., MAP is the etiological agent of the disease or condition).

### EXAMPLE 3

### Inhibition of MAP Proliferation with Salicylic Acids and a Sulfonamide

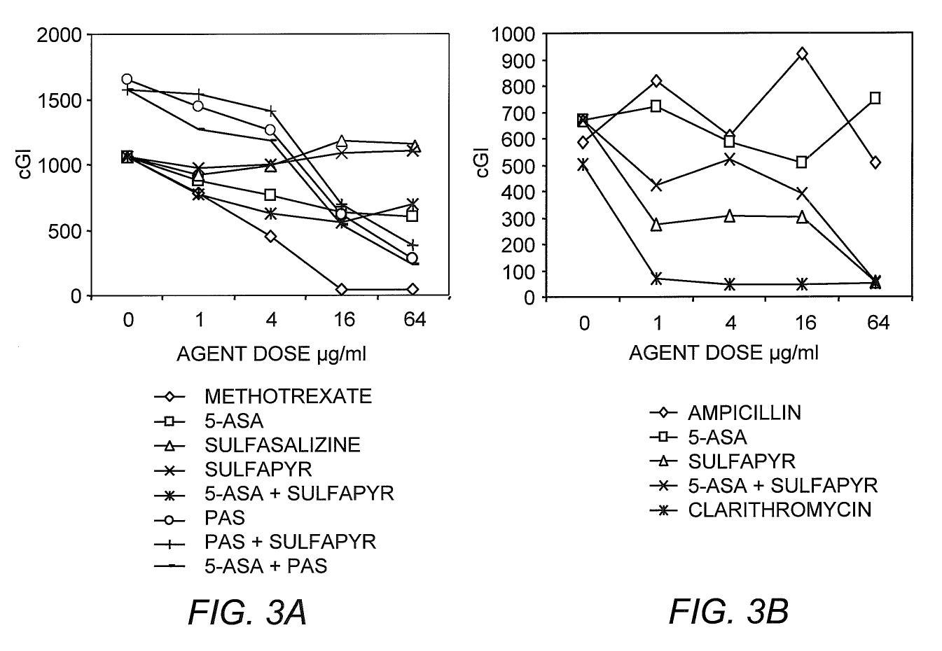

To demonstrate the efficacy of salicyclic acids and a sulfonamide folate antagonist in the inhibition of MAP proliferation, 5-ASA, p-aminosalicylic acid, and sulfapyridine were evaluated as individual compounds. Moreover, combinations of agents were tested as was the 5-ASA and sulfapyridine conjugate, sulfasalazine. As shown in FIG. 3A, 5-ASA and p-aminosalicylic acid inhibited the proliferation of MAP (ATCC 19698) in a dose-dependent manner, whereas sulfasalazine and sulfapyridine had little effect on the cumulative growth index of MAP. On the other hand, FIG. 3B shows that sulfapyridine and 5-ASA+sulfapyridine inhibited M. avium 101 in a dose-dependent manner, whereas 5-ASA had little effect.

Accordingly, these data demonstrate that there may be differences in the response of individual MAP isolates (e.g., isolated from different patients) to anti-MAP agents. For example, it is expected that MAP strains, ATCC 43015, ATCC 43544, ATCC 43545, ATCC 49164, ATCC 70053, K10 and JTC303, as well as clinical isolates from both veterinary and human sources, will respond differently to different individual compounds and combinations of compounds (sulfapyridine and 5-ASA). Therefore, as disclosed herein, it will be essential to determine the antibiotic susceptibility of a MAP isolated from a subject in order to select an appropriate anti-MAP agent, or combination of anti-MAP agents, to effectively treat a MAP infection.

### EXAMPLE 4

### Inhibition of MAP Proliferation with Thalidomide

Racemic thalidomide (±), as well as the (+) and (−) enantiomers, were analyzed for anti-MAP activity. In addition, MAP growth kinetics were determined in the presence of phthalimide and 1-Hydroxy-2,6-piperidinedione (HPD). This analysis was carried out using a human (Dominic) and bovine (ATCC 19698) strain of MAP and two strains of M. avium (ATCC 25291 and 101.) A radiometric (¹⁴CO₂ BACTEC®) detection system was used to quantify mycobacterial growth as arbitrary “growth index units” (GI.) Efficacy data are presented as “percent decrease in cumulative GI” (%−ΔcGI.)

The results of this analysis indicated that phthalimide did not exhibit a dose-dependent inhibition of any strain. Furthermore, there was no dose-dependent inhibition on either M. avium subspecies avium strain with thalidomide (Table 2), phthalimide or HPD. In contrast, dose-dependent inhibition was observed for thalidomide (+) (31%−ΔcGI for Dominic and 26%−ΔcGI for ATCC 19698 at 64 μg/ml thalidomide; Table 2), with thalidomide (+)(Table 3) being more inhibitory than thalidomide (−). HPD was, on a weight-for-weight basis, the most potent inhibitory agent evaluated (46%−ΔcGI for Dominic and −44%−ΔcGI for ATCC 19698 at 64 μg/ml HPD; Table 4).

TABLE 2

| | %-ΔcGI | | | | |
|---|---|---|---|---|---|
| Thalidomide | MAP | | | | |
| (±) | Dominic | | | M. avium | |
| (μg/ml) | Expt A | Expt B | ATCC 19698 | ATCC 25291 | 101 |
| 1 | −2% | 0% | 4% | 34% | −20% |
| 4 | −4% | −2% | 8% | 37% | −5% |
| 16 | −9% | −5% | −7% | 28% | 6% |
| 64 | −31% | −13% | −26% | 25% | −14% |

TABLE 3

| | |
|---|---|
| Thalidomide | %-ΔcGI |

| (+) (μg/ml) | MAP (Dominic) | M. avium (101) |
|---|---|---|
| 1 | −4% | 4% |
| 4 | 1% | −5% |
| 16 | −14% | 16% |
| 64 | −58% | −19% |

TABLE 4

| | %-ΔcGI | | | | |
|---|---|---|---|---|---|
| | MAP | | | | |
| HPD | Dominic | | | M. avium | |
| (μg/ml) | Expt A | Expt B | ATCC 19698 | ATCC 25291 | 101 |
| 1 | −5% | 16% | 22% | −4% | −12% |
| 4 | −7% | 6% | 7% | 47% | −52% |
| 16 | −18% | 12% | −3% | 22% | −21% |
| 64 | −46% | −19% | −44% | 21% | −36% |

### EXAMPLE 5

### Inhibition of MAP Proliferation with Immunosuppressants

Known immunosuppressive agents were also analyzed for anti-MAP activity. Specifically, Cyclosporine A, Tacrolimus (FK506) and Rapamycin were analyzed for anti-MAP activity against both human and bovine strains of MAP, M. avium subspecies avium and BCG (as a level II surrogate for M. tuberculosis). Six strains of mycobacteria were analyzed, four of which were MAP. Two MAP strains were isolated from humans with Crohn's disease: Dominic (ATCC 43545; Chiodini, et al. (1986) J. Clin. Microbiol. 24:357-363) and UCF 4 (Naser, et al. (2004) Lancet 364:1039-1044). The other two MAP strains were from ruminants with Johne's disease ATCC 19698 (ATCC, Rockville, Md.) and 303 (Sung & Collins (2003) Appl. Environ. Microbiol. 69(11):6833-6840). The M. avium subspecies avium strains (hereinafter called M. avium) were ATCC 25291 (veterinary source) and M. avium 101 (Betram, et al. (1986) J. Infect. Dis. 154:194-195).

Because it renders clinically resistant strains of MAP inappropriately susceptible to antimicrobials in cell culture (Damato & Collins (1990) Vet. Microbiol. 22:31-42), TWEEN 80 detergent was not included in the cultures. Prior to inoculation, cultures were processed as described herein and according to conventional methods (Rastogi, et al. (1992) Antimicrobiol. Agents Chemother. 36:2843-2846).

For experimental comparability, only chemicals that could be solubilized with DMSO were used. The positive control antibiotics were clofazimine, an antibiotic used to treat leprosy (Britton & Lockwood (2004) Lancet 363:1209-1219) and in clinical trials for the treatment of Crohn's disease (Selby, et al. (2007) Gastroenterology 132:2313-2319; Borody, et al. (2002) Dig. Liver Dis. 34:29-38), and monensin, an antibiotic used in veterinarian medicine (McDougald (1976) Poult. Sci. 55:2442-2447; Melendez, et al. (2006) Prev. Vet. Med. 73:33-42). The two negative controls were gluterimide antibiotics, cycloheximide and phthalimide.

The tested agents, Cyclosporine A, Rapamycin and Tacrolimus (all Sigma, St. Louis, Mo.) were solubilized in 100% DMSO. Aliquots were prediluted, stored at −80° C. in 50% DMSO (Sigma) in water, thawed, used once and discarded. Volumes of DMSO were adjusted so that final concentration in every BACTEC® vial used was always 3.2% DMSO. Agents were tested in serial dilutions from a minimum of 0.5 μg/ml to a maximum of 64 μg/ml. Inhibition of mycobacterial growth is expressed as % −ΔcGI, and enhancement as % +ΔcGI compared to 3.2% DMSO controls. In other words, a negative % ΔcGI is the percent decrease in cumulative GI compared to control inoculation.

Data for individual chemical agents are presented in Tables 5-9. The positive experimental controls were clofazimine (Table 5) and monensin (Table 6). The “negative” controls were cycloheximide (Table 7) and phthalimide (Table 8). Tables 9-11 present data for Cyclosporine A, Rapamycin, and Tacrolimus, respectively.

TABLE 5

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Clofazimine | Dominic | Dominic | | Bovine MAP | | Bovine | |
| (μg/ml) | Expt. 1 | Expt. 2 | UCF4 | 303 | 19698 | 25291 | 101 |
| 1 | −99 | −99 | −99 | −99 | −99 | −98 | −98 |
| 4 | −99 | −99 | −99 | −99 | −99 | −98 | −98 |
| 16 | −99 | −99 | −99 | −99 | −99 | −98 | −98 |
| 64 | −99 | −99 | −99 | −99 | −99 | −99 | −99 |

TABLE 6

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Monensin | Dominic | Dominic | | Bovine MAP | | Bovine | |
| (μg/ml) | Expt. 1 | Expt. 2 | UCF4 | 303 | 19698 | 25291 | 101 |
| 1 | −52 | −46 | −73 | −21 | −14 | −25 | 7 |
| 4 | −77 | −71 | −93 | −73 | −45 | −54 | 3 |
| 16 | −93 | −87 | −96 | −94 | −62 | −69 | 4 |
| 64 | −94 | −92 | −97 | −97 | −65 | −87 | 8 |

TABLE 7

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Cycloheximide | Dominic | Dominic | | Bovine MAP | | Bovine | |
| (μg/ml) | Expt. 1 | Expt. 2 | UCF4 | 303 | 19698 | 25291 | 101 |
| 1 | 15 | 9 | 1 | 3 | −2 | 0 | 6 |
| 4 | −4 | 0 | −7 | 4 | −7 | −8 | 7 |
| 16 | −8 | −4 | −4 | −5 | −12 | −7 | 1 |
| 64 | −1 | −7 | −12 | −4 | −9 | −57 | 5 |

TABLE 8

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Phthalimide | Dominic | Dominic | | Bovine MAP | | Bovine | |
| (μg/ml) | Expt. 1 | Expt. 2 | UCF4 | 303 | 19698 | 25291 | 101 |
| 1 | −3 | −1 | 4 | −2 | −2 | 2 | −3 |
| 4 | 7 | 0 | −2 | 3 | −10 | 4 | 4 |
| 16 | 0 | 1 | 1 | −13 | 1 | 1 | 2 |
| 64 | 2 | 4 | 3 | 6 | 0 | 12 | −2 |

TABLE 9

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Cyclosporin | Dominic | Dominic | | Bovine MAP | | Bovine | |
| A (μg/ml) | Expt. 1 | Expt. 2 | UCF4 | 303 | 19698 | 25291 | 101 |
| 1 | 15 | 4 | −2 | −1 | −2 | 22 | −28 |
| 4 | −10 | −9 | 5 | −3 | −23 | 31 | −44 |
| 16 | −43 | −64 | −9 | −14 | −19 | 4 | −56 |
| 64 | −98 | −99 | −99 | −91 | −92 | −54 | −95 |

TABLE 10

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Rapamycin | Dominic | Dominic | | Bovine MAP | | Bovine | |
| (μg/ml) | Expt. 1 | Expt. 2 | UCF4 | 303 | 19698 | 25291 | 101 |
| 1 | 21 | 0 | −14 | 4 | −3 | 10 | −19 |
| 4 | 13 | −7 | −9 | −11 | −1 | 7 | −7 |
| 16 | −10 | −9 | −29 | −15 | −18 | 11 | −28 |
| 64 | −58 | −44 | −76 | −39 | −43 | −18 | −39 |

TABLE 11

| | Mycobacterial strain (% ΔcGI) | | | | | | |
|---|---|---|---|---|---|---|---|
| | M. avium subspecies paratuberculosis | | | | | | |
| | Human MAP | | | | | M. avium | |
| Tacrolimus | Dominic | Dominic | | Bovine MAP | | Bovine | |
| (μg/ml) | Expt. 1 | Expt. 2 | UCF4 | 303 | 19698 | 25291 | 101 |
| 1 | 28 | 5 | 6 | 5 | 8 | −23 | −34 |
| 4 | 9 | 9 | −19 | 3 | −3 | −3 | −43 |
| 16 | 21 | −10 | −18 | −5 | −5 | 11 | −40 |
| 64 | 0 | −21 | −43 | −27 | −26 | 53 | −52 |

The most potent positive control was clofazimine, 97% −ΔcGI at 0.5 (Dominic; Table 5). The other positive control, Monensin, had dose-dependent inhibition against all MAP strains. It was most effective against MAP isolated from humans (Table 6) and bovine 303, and least against bovine ATCC 25291 (Table 6). Monensin had no inhibition against M. avium 101 (Table 6).

The negative control chemical agents were the gluterimide antibiotics cycloheximide and phthalimide. Cycloheximide had no dose-dependent inhibition on any MAP strain (Table 7). Cycloheximide had dose-dependent inhibition on M. avium ATCC 25291, (57% −ΔcGI at 64 μg/ml) but no affect on M. avium 101 (Table 7). Phthalimide had no dose-dependent effect on any strain tested (Table 8).

The three “immunosuppressants” tested were Cyclosporine A, Rapamycin and Tacrolimus. There were differing amounts of inhibition depending on the agent and strain. The control mycobacterial strains were M. avium subspecies avium ATCC 25291 and 101. Of the three “immunosuppressants,” Cyclosporine A had dose-dependent inhibition on M. avium subspecies avium 101 (95% −ΔcGI at 64 μg/ml) (Table 9). There was no inhibition with Rapamycin or Tacrolimus on the control M. avium 25291 (Tables 10 and 11).

Against MAP, Cyclosporine A was the most effective of the three “immunosuppressants” studied. On MAP isolated from humans, (Dominic and UCF 4), Cyclosporine had 97% −ΔcGI at 32 μg/ml against Dominic and 99% −ΔcGI at 64 μg/ml on Dominic and UCF 4 (Table 9). On MAP isolated from ruminants, Cyclosporine A had slightly less dose-dependent inhibition (ATCC 19698: 92% −ΔcGI at 64 μg/ml) than against MAP isolated from humans (Table 9). Rapamycin was the second most effective “immunosuppressant” studied. At lower concentrations (1 and 16 μg/ml) Rapamycin had no inhibition and at 64 μg/ml it had 76% −ΔcGI on UCF 4, a MAP isolated from humans (Table 10). Rapamycin was less effective against MAP isolated from ruminants and had no effect on M. avium ATCC 25291 (Table 10). Tacrolimus had the least inhibition of the three “immunosuppressants” studied. Against MAP, Tacrolimus was most inhibitory against UCF 4 (43% −ΔcGI at 64 μg/ml) and ATCC 19698 (26% −ΔcGI at 64 μg/ml) (Table 11). Paradoxically, Tacrolimus exhibited the most inhibition on M. avium 101 of all six strains studied, yet actually enhanced growth of M. avium ATCC 25291. (Table 11).

Rapamycin was initially evaluated as an anti-fungal agent (Singh, et al. (1979) J. Antibiot. (Tokyo) 32:630-654). However, this is the first time that anti-MAP activity has been demonstrated for the “immunosuppressant” agents Cyclosporine A, Rapamycin and Tacrolimus. These observations therefore further demonstrate MAP is responsible for multiple “autoimmune” and “inflammatory” diseases, and that the action of these three “immunosuppressant” agents may simply be to inhibit MAP growth.

As with the “high” and “low” doses of methotrexate and 6-MP used in the treatment of human malignancies and “autoimmune” or “inflammatory” conditions, respectively, there are “high” and “low” doses of the three “immunosuppressants” studied herein, wherein “low” doses have prokaryotic antibiotic action in addition to eukaryotic immunosuppressant activity. The data presented herein are subtle and the negative controls are critical. It must be emphasized that these data were obtained using the exquisitely sensitive radiometric ¹⁴C Bactec® system. As with 5-ASA, the effects of Cyclosporine A, Rapamycin and Tacrolimus may not be detectable using the more convenient, fluorescent-based MIGT System®.

What is claimed is:

1. A method for inhibiting the proliferation of Mycobacterium avium subspecies paratuberculosis (MAP) comprising contacting MAP with an effective amount of at least two anti-MAP agents, or a prodrug or metabolite thereof, selected from the group consisting of a folate antagonist, a purine inhibitor, a thymidylate synthase inhibitor, an antibiotic, an immunosuppressant and a thalidomide, so that proliferation of the MAP is inhibited.

2. The method of claim 1, further comprising administering a salicylic acid.

3. A method for inhibiting the proliferation of Mycobacterium avium subspecies paratuberculosis (MAP) comprising contacting a MAP with an effective amount of a salicylic acid so that proliferation of the MAP is inhibited.

4. The method of claim 3, further comprising administering a folate antagonist.

5. A method for treating a Mycobacterium avium subspecies paratuberculosis (MAP) infection comprising administering to a subject having or at risk of having a MAP infection an effective amount of at least two anti-MAP agents, or a prodrug or metabolite thereof, selected from the group consisting of a folate antagonist, a purine inhibitor, a thymidylate synthase inhibitor, an antibiotic, an immunosuppressant and a thalidomide so that the MAP infection is treated.

6. The method of claim 5, further comprising administering a salicylic acid.

7. A method for treating a Mycobacterium avium subspecies paratuberculosis (MAP) infection comprising administering to a subject having or at risk of having a MAP infection an effective amount of a salicylic acid so that the MAP infection is treated.

8. The method of claim 7, further comprising administering a folate antagonist.

9. A pharmaceutical composition comprising at least two anti-Mycobacterium avium subspecies paratuberculosis (anti-MAP) agents, or a prodrug or metabolite thereof, selected from the group consisting of a folate antagonist, a purine inhibitor, a thymidylate synthase inhibitor, an antibiotic, an immunosuppressant and a thalidomide, in admixture with a pharmaceutically acceptable carrier.

10. A pharmaceutical composition comprising a salicylic acid and a folate antagonist in admixture with a pharmaceutically acceptable carrier.

11. A method for diagnosing Multiple Sclerosis or Alzheimer's Disease comprising detecting the presence of a Mycobacterium avium subspecies paratuberculosis (MAP) nucleic acid molecule in a sample from a subject having or suspected of having Multiple Sclerosis or Alzheimer's Disease, wherein the presence of MAP nucleic acid molecule in the sample is indicative of Multiple Sclerosis or Alzheimer's Disease.

12. The method of claim 11, further comprising the prestep of in vitro culturing of Mycobacterium avium subspecies paratuberculosis in the sample.

13. A method for determining the antibiotic susceptibility of Mycobacterium avium subspecies paratuberculosis (MAP) isolated from a subject comprising growing MAP from a sample under suitable conditions to obtain the cell wall containing form of MAP and contacting the MAP with a plurality of antibiotics to determine the antibiotic susceptibility of the MAP.

14. A method for inhibiting the proliferation of Mycobacterium avium subspecies paratuberculosis (MAP) comprising contacting MAP with an effective amount of an immunosuppressant so that proliferation of the MAP is inhibited.

15. The method of claim 14, wherein the immunosuppressant is a cyclosporine or a macrolide immunosuppressant.
